(19) United States
(12) Plant Patent Application Publication
(10) Pub. No.: US 2003/0213040 P1
(43) Pub. Date: Nov. 13, 2003

(54) Variety of geranium plant named 'Penlava'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/739,023

(22) Filed: Dec. 8, 2000

(51) Int. Cl. A01H 5/00

(52) U.S. Cl. PLT/324

(57) ABSTRACT

A new and distinct zonal geranium plant with intense purple colored flowers above medium green foliage. The plant flower is suitable for pot planting and as a bedding plant.

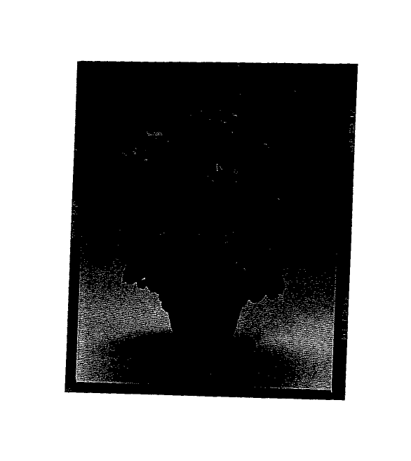

### BACKGROUND OF THE INVENTION

The present invention comprises a new and distinct cultivar of Pelargonium×hortorum known by the varietal name ‘Penlava’. The new variety was discovered in 1994 in a selective breeding program in Dresden, Germany. The new variety is the result of the crossing of dark leafed breeding varieties. The new variety was first asexually reproduced by cuttings in Dresden, Germany in 1995. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to the R.H.S. Colour Chart of The Royal Horticultural Society of London.

### PLANT

Classification:

Botanical.—Pelargonium×hortorum.

Commercial.—Zonal geranium.

Form: Erect.

Height from media surface to top of foliage: 40 cm.

Height from media surface to top of umbel: 50 cm.

Growth: Medium degree of branching.

Strength: Does not require artificial support.

Response time: 3.5 weeks.

Leaves:

Size.—8-10 cm long.

Shape.—Cordate.

Margin.—Partially toothed.

Texture.—Hairy.

Color.—Green Group 139A with weak zone.

Ribs and veins.—Color: Brighter than leaf surface.

Petioles:

Color.—Medium green.

Length.—6-8 cm.

Stem/branches:

Color.—Medium green.

Internode length.—4-6 cm.

### THE BUD

Shape when just showing color:

Overall.—Spherical.

Individual bud.—Elliptical.

Color: Green Group 136B.

Size when just showing color:

Umbel.—8-10 cm across.

Individual bud.—1.2 cm long; 0.6-0.8 cm wide.

### INFLORESCENCE

Blooming habit: Semi-double.

Size of fully open bloom: 4.5 cm across.

Borne: Flowers on umbel; umbel on pedicel; pedicel on peduncle.

Flowers:

Form.—Zygomorph.

Number of petals.—7-9.

Size.—2.0-2.5 cm across.

Petals.—Color: Upper surface: Purple Group 78C. Lower surface: Purple Group 75C.

Texture and appearance.—Velvety.

Petaloids: Absent.

Pedicel:

Length.—2.0-2.5 cm.

Color.—Reddish-green.

Peduncle:

Length.—10 cm.

Color.—Reddish-green.

Disease/pest resistance: No unusual susceptibility to diseases or pests has been noted to date.

Lasting Quality: Long-lasting; 4 weeks per umbel.

### REPRODUCTIVE ORGANS

Stamens:

Anthers.—3 mm long.

Filaments.'Length: 0.5 cm maximum. Color: White.

Pollen.—Orange.

Pistils:

Number.—Five.

Length.—0.3-0.4 cm.

Stigma.—Pink.

Style.—0.8 cm long.

Ovaries:

Color.—Greenish.

Length.—0.4 cm.

Fruit: Very rarely present.

### GENERAL CHARACTERISTICS

1. Dark-leafed.

2. Intense flower color with eye.

3. Shiny flower color.

4. Vigorous growth habit.

I claim:

1. A new and distinct variety of geranium plant substantially as shown and described.
